(19) United States
(12) Patent Application Publication
(10) Pub. No.: US 2026/0021474 A1
(43) Pub. Date: Jan. 22, 2026

(54) USE OF PARTICLES OF TITANIUM DIOXIDE BEARING A METAL OR A METAL OXIDE FOR OBTAINING ALKENES BY PHOTOCATALYSIS

(71) Applicants: COMMISSARIAT A L'ENERGIE ATOMIQUE ET AUX ENERGIES ALTERNATIVES, Paris (FR); CENTRE NATIONAL DE LA RECHERCHE SCIENTIFIQUE, Paris (FR); UNIVERSITÉ CLAUDE BERNARD LYON I, Villeurbanne (FR)

(72) Inventors: Nathalie HERLIN, GIF-SUR-YVETTE CEDEX (FR); Juliette KARPIEL, GIF-SUR-YVETTE CEDEX (FR); Pierre LONCHAMBON, GIF-SUR-YVETTE CEDEX (FR); Chantal GUILLARD, VILLEURBANNE Cedex (FR); Frédéric DAPPOZZE, VILLEURBANNE Cedex (FR)

(73) Assignees: COMMISSARIAT A L'ENERGIE ATOMIQUE ET AUX ENERGIES ALTERNATIVES, Paris (FR); CENTRE NATIONAL DE LA RECHERCHE SCIENTIFIQUE, Paris (FR); UNIVERSITÉ CLAUDE BERNARD LYON I, Villeurbanne (FR)

(21) Appl. No.: 18/870,279

(22) PCT Filed: Jun. 2, 2023
(86) PCT No.: PCT/EP2023/064819

§ 371 Date: Aug. 5, 2025

(30) Foreign Application Priority Data
Jun. 3, 2022 (FR) 2205384

(51) Int. Cl. B01J 21/06; B01J 23/72; B01J 35/39; B01J 35/64; C07C 2/24; C07C 11/04

(52) U.S. Cl. CPC B01J 21/063; B01J 23/72; B01J 35/39; B01J 35/643; B01J 35/647; B01J 35/651; C07C 2/24; C07C 11/04

(57) ABSTRACT

This invention concerns the use of TiO₂ particles bearing a metal and/or a metal oxide to obtain alkenes by photocatalysis. This invention also covers a method for obtaining alkenes by photocatalysis of carboxylic acids and/or alcohols in the presence of a catalyst based on TiO₂ particles bearing a metal and/or a metal oxide.

This invention concerns the use of TiO₂ particles bearing a metal and/or a metal oxide to obtain alkenes by photocatalysis. This invention also covers a method for obtaining alkenes by photocatalysis of carboxylic acids and/or alcohols in the presence of a catalyst based on TiO₂ particles bearing a metal and/or a metal oxide.

Alkenes are generally produced during the refining of crude oil, and are compounds widely used in the chemical industry for many different applications, including as raw materials for the production of polymers such as plastics and lacquers, as well as for the synthesis of alcohols, surfactants and fuels.

Among the alkenes, ethylene is the most widely produced and used organic molecule in the world. The basis of the petrochemical industry, its global market is worth more than 130 billion dollars each year, with a consumption exceeding 150 million tons per year. These high tonnages may be explained by the fact that ethylene is the basic monomer used in the manufacture of over 75% of petrochemical products. It is mainly used in the synthesis of plastics such as polyethylene (PET), or in the synthesis of chemical surfactants such as ethylene oxide and ethylene glycol.

Currently, 99% of industrial ethylene production is based on the cracking of naphtha (an intermediate between petrol and kerosene, produced by fractional distillation of petroleum) or hydrocarbons such as ethane, usually heated to between 750 and 950° C. The yields of ethylene are variable, around 35% from naphtha and 80% from ethane. However, as well as being very energy-intensive, this synthesis is polluting and dependent on oil resources. With fossil fuels running out and the climate challenge looming, it has become vital to develop new low-energy, low-cost ethylene synthesis methods based on renewable resources.

Given these challenges, a great deal of research has been implemented to find an alternative to cracking petroleum derivatives to produce ethylene.

One of these alternative technologies is the catalytic dehydration of ethanol into ethylene. One example is bioethanol, a renewable resource produced at low cost without the use of toxic reagents. The ethanol dehydration uses acid catalysts such as monofunctional oxides, typically γ-Al₂O₃ alumina, or “molecular sieves” such as zeolitic structures like ZSM-5 (Zeolite Socony Mobil-5). However, despite the use of acid catalysts, the ethanol dehydration reaction remains highly endothermic and only shifts to ethylene at high temperature. The working temperature and pressure therefore have a significant effect on ethylene yield. As a result, these technologies generally allow high ethylene yields to be obtained, but the temperatures (typically between 300 and 550° C.) and the pressures (generally between 0.3 and 4 MPa) employed remain high, and are therefore energy-intensive and costly. And generally, when the working temperatures are lower, the formation of a non-negligible quantity of by-product is observed, with a limited percentage of ethanol conversion and pressures remaining high. In addition, the acid catalysts typically used to lower the working temperature have a limited service lifetime because they tend to deactivate by coking (formation by thermal cracking of a deposit of coke on the surfaces of a system subjected to high temperatures, with the result of reducing its performance), and the regeneration reactions at high temperatures lead to high costs and a significant loss of catalytic efficiency.

Other technologies, based on the production of ethylene by photocatalysis, have subsequently been developed, using CuCl₂ as the catalyst. These methods are interesting because they use light energy instead of thermal energy and high pressure. However, the CuCl₂-type catalyst becomes inactive very quickly, typically in less than an hour, and must therefore be regularly regenerated in air.

In addition to ethanol, carboxylic acids, in particular propanoic acid (CH₃CH₂COOH), are of particular interest because they may be used as renewable resources within biomass to produce liquid fuels and other molecules of interest to the energy sector. The propanoic acid is a volatile fatty acid found as a pollutant in domestic wastewater. It may also be obtained from biomass by fermentation of glycerol, as well as from fermentation of industrial water from, for example, the extraction of rapeseed oil or waste derived from potatoes.

Methods for the decarboxylation of propanoic acid into alkanes by photocatalysis using TiO₂ powders, which may be platinized, have been developed in this context. Ethylene may also be formed using these methods, but only in trace amounts as a result of secondary reactions. Furthermore, the platinum used in certain methods is a noble platinum metal, which makes it an expensive material to produce and therefore unsuitable for development on an industrial scale.

The aim of the present invention is to provide a method for obtaining alkenes, in particular ethylene, by a photocatalysis reaction which avoids the aforementioned disadvantages. Thus, one aim of the invention is to provide a method for producing alkene or alkenes, in particular ethylene, which is free from fossil resources and does not require significant heating (i.e. typically above 200° C.), or even the input of thermal energy. None of the solutions currently proposed may achieve both a good yield and/or selectivity in alkene or alkenes, in particular ethylene, under ambient temperature and pressure conditions, and in particular on volumes compatible with the industrial scale of alcohol or acid solutions that may be synthesized from biomass.

Another aim of the invention is to provide a method that is simple to implement, stable over time (for example for at least 50 hours) and inexpensive. Indeed, some approaches in the prior art propose the use of expensive photocatalysts based on noble metals and/or which deactivate rapidly under irradiation and therefore need to be regenerated, generating additional costs.

Another aim of the invention is to provide a method for obtaining alkenes, in particular ethylene, with an excellent selectivity, particularly at ambient temperature and pressure, under simple UV/visible irradiation.

Thus, according to a first aspect, the invention relates to the use of particles consisting of or comprising TiO₂ bearing on at least part of their surface a metal M and/or an oxide of metal M, M being chosen from the group comprising Cu, Zn, Fe, Mo, W and Ni, and more particularly from the group comprising Cu, Zn, Fe and Ni, for obtaining at least one alkene by photocatalysis from at least one carboxylic acid of formula (I) Rₐ—COOH, and/or at least one alcohol of formula (II) R_{b}—OH, wherein Rₐ and R_{b} are independently chosen from linear, branched or cyclic alkyl groups, in particular linear, branched or cyclic C₂ to C₁₈ alkyl groups, more particularly linear, branched or cyclic C₂ to C₆ alkyl groups, said alkyl groups optionally being substituted with at least one group X chosen from arenes, X in particular being a phenyl group.

“Photocatalysis” refers in particular to a reaction catalyzed by the action of light rays on the surface of a catalyst called a photocatalyst.

According to a particular embodiment, the largest number-average dimension of the particles is between 1 and 100 nm, in particular between 5 and 70 nm. The largest number-average dimension may be measured by any technique known to the person skilled in the art, in particular by size measurement by counting, for example using the ImageJ software, on transmission electron microscopy (TEM) images.

In a particular embodiment, the particles consisting of or comprising TiO₂ are spherical, spheroidal, rod-shaped, wire-shaped, tubular and/or platelet-shaped. These comprise nanospheres, nanospheroids, nanorods, nanowires, nanotubes and/or nanoplatelets. The particles consisting of or comprising TiO₂, in particular in one of the forms described above, may optionally be organized into chains, in particular nanochains.

According to a particular embodiment, the TiO₂ is in the form of anatase, rutile and/or brookite, in particular in the form of anatase, rutile, or a mixture of anatase and rutile, more particularly in the form of a mixture of anatase and rutile whose anatase/rutile ratio is between 0.80 and 2.33, in particular between 1.00 and 2.33, more particularly between 1.00 and 2.00.

In a particular embodiment, the particles consisting of or comprising TiO₂ have a specific surface area comprised between 10 and 500 m²/g, in particular between 30 and 150 m²/g. These ranges of specific surface area may correspond to the specific surface area of the particles consisting of or comprising TiO₂ excluding the surface area corresponding to the metal M and/or oxide of metal M. These ranges of specific surface area may correspond to the total specific surface area of the particles consisting of or comprising TiO₂ bearing the metal M and/or the oxide of metal M.

According to a particular embodiment, the content of metal M and/or oxide of metal M relative to TiO₂ is from 0.01 to 50% by mass, in particular from 0.1 to 5% by mass, for example approximately 2% by mass, or more than 0.01% and less than 2% by mass. According to a particular embodiment, the content of metal M and/or oxide of metal M relative to TiO₂ is from 0.01 to 1.0; 1.2; 1.4; 1.6; 1.8 or 1.9% by mass, in particular from 0.1 to 1.0; 1.2; 1.4; 1.6; 1.8 or 1.9% by mass.

According to a particular embodiment, the particles consisting of or comprising TiO₂ bearing on at least part of their surface a metal M and/or an oxide of metal M, also comprise, within them, the metal M and/or the oxide of metal M.

In a particular further embodiment, the majority of the metal M and/or the oxide of metal M of particles consisting of or comprising TiO₂ is present on the surface of said particles.

According to another particular embodiment, more than 50% by mass, in particular more than 60, 70, 75, 80, 85, 90, 91, 92, 93, 94, 95, 96, 97, 98 or 99% by mass of the metal M and/or the oxide of metal M of the particles consisting of or comprising TiO₂ is present on the surface of said particles. This may be measured by any technique known to the person skilled in the art, for example by comparing the measurements made by inductively coupled plasma spectrometry (ICP), which allow to quantify the total metal content of a material, with those made by X-ray photoelectron spectrometry (XPS), which allow to measure the quantity of surface elements in the material.

According to a particular embodiment, the metal M and/or the oxide of metal M are present, at least on the surface of the particles consisting of or comprising TiO₂, in the form of particles whose largest number-average particle size is between 0.1 and 50 nm, in particular between 0.5 and 10 nm, more particularly between 1 and 3 nm.

According to a particular embodiment, the particles consisting of or comprising TiO₂ bearing on at least part of their surface a metal M and/or an oxide of metal M are obtained by laser pyrolysis or by impregnation, optionally followed by annealing, in particular in air, in particular at a temperature of 300 to 500° C., in particular 400 to 500° C., for example at a temperature of approximately 450° C., and/or in particular for 3 hours, in particular 3 to 6 hours.

In a particular embodiment, the particles of the invention do not comprise gold.

According to a particular embodiment, the invention relates to a method for obtaining at least one alkene from at least one carboxylic acid, which is in particular propanoic acid, acetic acid, a phenylpropanoic acid, in particular 2-phenylpropanoic acid, n-butyric acid, n-valeric acid or pivalic acid, more particularly propanoic acid.

According to a particular embodiment, the invention concerns a method for obtaining at least one alkene from at least one alcohol, which is in particular ethanol, or cyclohexanol, more particularly ethanol.

In a particular embodiment, the at least one alkene is ethylene.

In a particular embodiment, the invention concerns a method for obtaining ethylene from propanoic acid.

According to another aspect, the invention also relates to a method for obtaining at least one alkene from at least one carboxylic acid of formula (I) Rₐ—COOH, and/or from at least one alcohol of formula (II) R_{b}—OH, wherein Rₐ and R_{b} are independently chosen from linear, branched or cyclic alkyl groups, optionally substituted with at least one group X chosen from arenes, X being in particular a phenyl group, comprising a step (i) of photocatalysis by UV and/or visible irradiation of at least one carboxylic acid and/or at least one alcohol in the presence of a catalyst consisting of or comprising particles consisting of or comprising TiO₂ bearing on at least part of their surface a metal M and/or an oxide of metal M, M being chosen from the group comprising Cu, Zn, Fe, Mo, W and Ni, and more particularly from the group comprising Cu, Zn, Fe and Ni.

According to a particular embodiment, the largest number-average dimension of the particles is between 1 and 100 nm, in particular between 5 and 70 nm. The largest number-average dimension may be measured by any technique known to the person skilled in the art, in particular by size measurement by counting, for example using the ImageJ software, on transmission electron microscopy (TEM) images.

In a particular embodiment, the particles consisting of or comprising TiO₂ are spherical, spheroidal, rod-shaped, wire-shaped, tubular and/or platelet-shaped. These comprise nanospheres, nanospheroids, nanorods, nanowires, nanotubes and/or nanoplatelets.

The particles consisting of or comprising TiO₂, in particular in one of the forms described above, may optionally be organized into chains, in particular nanochains.

According to a particular embodiment, the TiO₂ is in the form of anatase, rutile and/or brookite, in particular in the form of anatase, rutile, or a mixture of anatase and rutile, more particularly in the form of a mixture of anatase and rutile whose anatase/rutile ratio is between 0.80 and 2.33, in particular between 1.00 and 2.33, more particularly between 1.00 and 2.00.

In a particular embodiment, the particles consisting of or comprising TiO₂ have a specific surface area comprised between 10 and 500 m²/g, in particular between 30 and 150 m²/g. These ranges of specific surface area may correspond to the specific surface area of the particles consisting of or comprising TiO₂ excluding the surface area corresponding to the metal M and/or oxide of metal M. These ranges of specific surface area may correspond to the total specific surface area of the particles consisting of or comprising TiO₂ bearing the metal M and/or the oxide of metal M.

According to a particular embodiment, the content of metal M and/or oxide of metal M relative to TiO₂ is between 0.01 to 50% by mass, in particular between 0.1 to 5% by mass, for example approximately 2% by mass, or more than 0.01% and less than 2% by mass.

According to a particular embodiment, the content of metal M and/or oxide of metal M relative to TiO₂ is from 0.01 to 1.0; 1.2; 1.4; 1.6; 1.8 or 1.9% by mass, in particular from 0.1 to 1.0; 1.2; 1.4; 1.6; 1.8 or 1.9% by mass.

According to a particular embodiment, the particles consisting of or comprising TiO₂ bearing on at least part of their surface a metal M and/or an oxide of metal M, also comprise, within them, the metal M and/or the oxide of metal M.

In a particular further embodiment, the majority of the metal M and/or the oxide of metal M of particles consisting of or comprising TiO₂ is present on the surface of said particles.

According to another particular embodiment, more than 50% by mass, in particular more than 60, 70, 75, 80, 85, 90, 91, 92, 93, 94, 95, 96, 97, 98 or 99% by mass of the metal M and/or the oxide of metal M of the particles consisting of or comprising TiO₂ is present on the surface of said particles. This may be measured by any technique known to the person skilled in the art, for example by comparing the measurements made by inductively coupled plasma spectrometry (ICP), which allow to quantify the total metal content of a material, with those made by X-ray photoelectron spectrometry (XPS), which allow to measure the quantity of surface elements in the material.

According to a particular embodiment, the metal M and/or the oxide of metal M are present, at least on the surface of the particles consisting of or comprising TiO₂, in the form of particles whose largest number-average particle size is between 0.1 and 50 nm, in particular between 0.5 and 10 nm, more particularly between 1 and 3 nm.

According to a particular embodiment, the particles consisting of or comprising TiO₂ bearing on at least part of their surface a metal M and/or an oxide of metal M are obtained by laser pyrolysis or by impregnation, optionally followed by annealing, in particular in air, then optionally in dihydrogen, in particular at a temperature of 300 to 500° C., in particular 400 to 500° C., for example at a temperature of approximately 450° C., and/or in particular for 3 hours, in particular 3 to 6 hours.

The laser pyrolysis and the impregnation, for example, are carried out using methods well known to those person skilled in the art.

According to a more specific embodiment, the laser pyrolysis is performed by bringing an aerosol of a liquid composition comprising at least one TiO₂ precursor, at least one precursor of the metal M and/or of the oxide of metal M, and optionally an organic solvent, into contact with a laser beam.

According to another more specific embodiment, the impregnation is performed by bringing TiO₂, for example obtained by laser pyrolysis, into contact with a precursor of the metal M and/or of the oxide of metal M, the impregnation optionally being followed by annealing, in particular in air, then optionally in dihydrogen, in particular at a temperature of 300 to 500° C., in particular 400 to 500° C., for example at a temperature of approximately 450° C., and/or in particular for 3 hours, in particular 3 to 6 hours.

When the TiO₂ or TiO₂ bearing a metal M and/or an oxide of metal M on at least part of its surface is obtained by laser pyrolysis, annealing is carried out in air, then optionally in dihydrogen.

When TiO₂ is commercial and configured to be impregnated as defined above, annealing in air is optional but preferable, which may be followed advantageously by annealing in dihydrogen.

“Dihydrogen annealing” refers in particular to annealing under pure dihydrogen or diluted in an inert gas, such as argon or dinitrogen. If necessary, annealing may limit the presence of oxide on the surface of the metal M.

According to a particular embodiment, the invention relates to a method for obtaining at least one alkene from at least one carboxylic acid, which is in particular propanoic acid, acetic acid, a phenylpropanoic acid, in particular 2-phenylpropanoic acid, n-butyric acid, n-valeric acid or pivalic acid, more particularly propanoic acid.

According to a particular embodiment, the invention concerns a method for obtaining at least one alkene from at least one alcohol, which is in particular ethanol, or cyclohexanol, more particularly ethanol.

In a particular embodiment, the at least one alkene is ethylene.

In a particular embodiment, the invention concerns a method for obtaining ethylene from propanoic acid.

According to a particular embodiment, step (i) is performed in an atmosphere comprising less than 1% by volume of oxygen, in particular less than 0.1% oxygen, in particular less than 0.01% oxygen, more particularly less than 0.001% oxygen.

According to a particular embodiment, step (i) is performed under an inert gas atmosphere, in particular under a nitrogen, helium and/or argon atmosphere, more particularly under an argon atmosphere.

According to a more particular embodiment, the inert gas atmosphere is obtained by purging using a flow of inert gas, the flow being in particular from 1 to 500 mL/min, preferably from 50 to 70 mL/min.

According to a particular embodiment, step (i) is carried out under a continuous flow of inert gas, in particular under a nitrogen, helium and/or argon atmosphere, more particularly under an argon atmosphere, the flow being more particularly between 1 and 500 mL/min, preferably between 50 and 70 mL/min. In this case, the mode may be described as dynamic.

According to another particular embodiment, step (i) is carried out in the absence of a continuous flow of inert gas. In this case, the mode may be described as static. Still in this case, step (i) is performed in an inert gas atmosphere. In particular, a purge as defined above is carried out prior to step (i). This purge is then stopped before step (i) is carried out.

According to a particular embodiment, the at least one carboxylic acid and/or the at least one alcohol is present within a composition additionally comprising a solvent, in particular in solution in a solvent, the solvent preferably being water, the concentration of alcohol or alcohols and/or carboxylic acid or acids in the composition in particular being greater than or equal to 0.0001% by volume, in particular greater than or equal to 0.01% by volume, and/or less than 100% by volume, for example about 1.00% by volume.

According to another particular embodiment, the at least one carboxylic acid and/or the at least one alcohol is not in the presence of a solvent.

According to a particular embodiment, the catalyst is present in the composition comprising the at least one carboxylic acid and/or the at least one alcohol and the solvent, or, in the absence of solvent, in the at least one carboxylic acid and/or the at least one alcohol, at a concentration of between 0.01 and 50 g/L, for example about 0.5 g/L.

According to a particular embodiment, step (i) is performed at a temperature comprised from 10 and 200° C., in particular at a temperature of between approximately 20 and approximately 40° C., or at a temperature of between 40 and 200° C., in particular from 40 to 150° C., or even from 40 to 100° C.

According to a particular embodiment, step (i) is performed at a temperature of between 60 and 200° C., in particular at a temperature of between 60 and 150° C., especially between 60 and 100° C.

According to a particular embodiment, the irradiation is UV-A, UV-B, UV-C, and/or visible irradiation, in particular UV-A, in particular at a wavelength of between 350 and 400 nm.

According to a particular embodiment, the invention relates to a method comprising a step (ii), following step (i), of recovering the alkene or alkenes, this step (ii) optionally being followed by a step (iii) of isolating the alkene or alkenes.

Step (ii) may be performed by any technique known to the person skilled in the art, in particular by recovering the headspace of the photocatalytic device used.

Step (iii) may be performed by any technique known to the person skilled in the art, in particular by distillation, in particular by cryogenic distillation. This purification technique, based on the fact that each gas has its own boiling point, involves separating a gas mixture by varying the pressure and the temperature of the gas storage medium. The gas mixture is first cooled to a low temperature (usually T<−50° C.). After cooling, the gases are liquefied and sent to a distillation column. The liquid is heated gradually, allowing the gases to be separated according to their boiling temperature.

Step (iii) may also be performed using absorption-based techniques. The separation is based on the principle that each gas has a particular affinity for absorbents such as zeolites, alumina or activated carbon, or for solvents such as methanolamine (MEA). The Pressure Swing Absorption (PSA) method is the best illustration of this technique. The separation occurs when the gas mixture comes into contact with the absorbent/solvent in a tank subsequently pressurized. The gas with the best affinity for the absorbent is trapped, while the other gaseous species pass through the system. The reservoir is regenerated by returning to atmospheric pressure, releasing the previously trapped gas.

Step (iii) may also be performed by membrane separation, still based on the principle of the affinity of gases for a membrane, allowing the gases to infiltrate more or less rapidly through the membrane. The membrane materials frequently encountered in the literature are varied, including microporous organic polymers, zeolites and ceramic- or metal-based materials. In a first tank, the gas mixture is brought into contact with a membrane located at the interface of a second tank. The different gases diffuse into the second tank using a pressure gradient, promoting the mass transport across the membrane separating the retentate from the permeate.

According to a particular embodiment, the molar selectivity of alkene, in particular ethylene, is greater than or equal to 40%, in particular greater than 45%, more particularly greater than 50%.

According to a more particular embodiment, the molar selectivity for alkene, in particular ethylene, obtained from at least one carboxylic acid of formula (I) Rₐ—COOH, is greater than or equal to 50%, in particular greater than 60%, more particularly greater than 70, 80 or 90%.

According to another more specific embodiment, the molar selectivity for alkene, in particular ethylene, obtained from at least one alcohol of formula (II) R_{b}—OH, is greater than or equal to 40%, in particular greater than 45%, more particularly greater than 50%.

In a particular embodiment, no acetone and/or ethyl acetate is formed at the end of the method.

According to a particular embodiment, the molar selectivity of by-product chosen from acetone and/or ethyl acetate is less than 1%, in particular less than 0.1%, more particularly less than 0.01%.

### Definitions

As used in this description, the term “about” refers to a range of values within ±10% of a specific value. For example, the term “about 20” comprises the values of 20±10%, i.e., the values of 18 to 22.

For the purposes of this description, the percentages refer to percentages by mass in relation to the total mass of the formulation, unless otherwise stated.

As understood here, the value ranges in the form of “x-y” or “from x to y” or “between x and y” include the bounds x and y as well as the integers between these bounds. For example, “1-5”, or “from 1 to 5” or “between 1 and 5” refer to the integers 1, 2, 3, 4 and 5. The preferred embodiments include each individual integer in the value range, as well as any sub-combination of those integers. For example, the preferred values for “1-5” may comprise the integers 1, 2, 3, 4, 5, 1-2, 1-3, 1-4, 1-5, 2-3, 2-4, 2-5, etc.

As used herein, the term “alkyl” designates a linear or branched chain alkyl group having the number of carbon atoms indicated before said term, in particular 2 to 6 carbon atoms, such as ethyl, propyl, isopropyl, butyl, isobutyl, sec-butyl, tert-butyl, pentyl, isoamyl, neopentyl, 1-ethylpropyl, 3-methylpentyl, 2,2-dimethylbutyl, 2,3-dimethylbutyl, hexyl, etc.

Thus, an expression such as “C1-C4 alkyl” designates an alkyl radical containing from 1 to 4 carbon atoms. The same applies to the term “alkane”.

The cycloalkyls are in particular alkyls (as defined above) comprising a ring. One example is cyclohexyl.

As used herein, the term “arene” refers to a substituted or unsubstituted mono- or bicyclic aromatic hydrocarbon ring system having 6 to 10 carbon atoms in the ring. The examples include the benzene and the naphthalene. The preferred arenes comprise the unsubstituted or substituted benzene and naphthalene. The definition of “arena” comprises condensed ring systems, comprising, for example, the ring systems wherein an aromatic ring is condensed to a cycloalkyl ring. The examples of such condensed ring systems comprise, for example, the indane, the indene and the tetrahydronaphthalene.

### FIGURES

FIG. 1 shows an example of a photocatalytic device that may be used to implement a use or a method according to the present invention.
1. UV lamp;
2. Photocatalytic reactor;
3. Bubbler;
4. Cooling water inlet;
5. Cooling water outlet;
6. Solution containing the photocatalyst and the precursor alcohol or precursors alcohols and/or carboxylic acid or acids;
7. Headspace containing the gases produced during the photocatalysis;
8. Injector connected to a gas chromatography (GC) equipment;
9. Mechanical stirrer;
10. Isolation valve;
11. Mass flow rate controller;
12. Gas inlet;
13. Valve;
14. Gas chromatography (GC) equipment coupled with a flame ionization detector (FID) and a helium ionization detector (Pulsed Discharge Helium Ionization Detector (PDHID)).

FIG. 2 is a graph showing the production of ethylene according to example 3, under argon, from the degradation of propanoic acid (1 vol %) under UVA with TiO₂ (reference outside the invention) and Cu/TiO₂ (invention) nanoparticles synthesized by laser pyrolysis.

FIG. 3 shows a graph representing the production of ethylene according to example 3 under argon from the degradation of propanoic acid (1 vol %) under UVA with TiO₂ nanoparticles (reference outside the invention) synthesized by laser pyrolysis and Cu_{IMP/}TiO₂ (invention) obtained by impregnation of the TiO₂ support.

FIG. 4 shows the products obtained by photo-oxidation of ethanol (1% m) under UVA, depending on the irradiation time, according to example 4.

FIG. 5 describes the molar selectivity of CₓH_{y}O_{z} products as obtained by photo-oxidation of ethanol (1% m) under UVA, according to example 4.

### EXAMPLES

### Example 1: Example of a Photocatalytic Device for Implementing a Use or a Method According to the Invention

The photocatalytic device (FIG. 1) consists of a 250-mL Pyrex airtight reactor containing 100 mL of aqueous solution, 150 mL of headspace, a glass mechanical stirrer and a bubbler (also made of glass) for supplying neutral gas. The neutral gas may be helium He, nitrogen N₂ or preferably argon Ar.

An 18W Phillips PLL UVA lamp delivering a power flux of 4.8 mW·cm⁻² was used as the light source, centered at 370 nm. The wavelengths emitted were between 350 and 400 nm. The concentration of alcohol or alcohols or carboxylic acid or acids in the solution is between 0.01 and 100% by volume, preferably 1.00%. The alcohols compounds and/or carboxylic acids may or may not be added as a mixture. The concentration of metal (oxide)/TiO₂ photocatalyst is between 0.01 and 50 g/L, preferably 0.5 g/L.

After the ambient air in the reactor has been completely purged by bubbling in neutral gas, the aqueous suspension comprising one or more alcohols and/or one or more carboxylic acids and the photocatalyst is irradiated with UVA. The photocatalytic reaction may take place under continuous flow of neutral gas (dynamic mode) or statically (without flow of neutral gas), preferably statically. Typically, the flow of neutral gas during purging and/or under irradiation is between 1 and 500 mL/min, preferably 50 and 70 mL/min.

The gases produced in the headspace of the reactor during photocatalysis are analyzed by gas chromatography using a flame ionization detector (FID) and a helium plasma detector (PDHID). In the case of a reaction in dynamic mode, the gases are transported by the flow of neutral gas; in the case of a reaction in static mode, the gases are transported by pumping.

### Example 2: Preparation of a Catalyst Allowing the Implementation of a Use or a Method According to the Invention

The metal and/or metal oxide may be brought into contact with the surface of the TiO₂ particles by any technique known to the person skilled in the art. For example, this could be a laser pyrolysis or impregnation technique.

### Synthesis by Laser Pyrolysis:

The TiO₂ (outside the invention) and metal (oxide)/TiO₂ photocatalysts of the invention may be synthesized by the laser pyrolysis technique, an example of which is given below using copper as the metal.

A liquid mixture comprising the titanium and copper precursors is inserted into an enclosure referred to as a “pyrosol” comprising a cooling device, a drive gas inlet and a piezoelectric pellet. Typically, the titanium precursor is titanium isopropylate (TTIP); the copper precursor may be, for example, copper acetylacetonate Cu(acac)₂. Optionally, the copper precursor may be dissolved beforehand in one or more organic solvents such as a mixture of o-xylene/ethyl acetate in a ratio of 6.5:3.5 by volume.

The composition of said mixture is shown in Table 1 below for a target copper content of 2.00 wt. % based on the mass of TiO₂.

TABLE 1

| Material | TTIP | Cu(acac)₂ | o-xylene/ethyl acetate (6.5:3.5) |
|---|---|---|---|
| TiO₂ (outside | 175 g | — | 150 mL |
| the invention) | | | |
| Cu/TiO₂ | 175 g | 4.134 g | 150 mL |

### Composition of the Precursor Mixture for Laser Pyrolysis

The liquid mixture of precursors is converted into an aerosol by actuating the piezoelectric pellet. Optionally, the mixture may be heated throughout the synthesis, over a range from 10 to 100° C. Preferably, the mixture is heated to 30° C.

The aerosol obtained is then conveyed into a confined reaction chamber in a neutral atmosphere via a carrier gas, which may be helium He, argon Ar or nitrogen N₂. Similarly, the confinement gas in the reaction chamber may be helium He, argon Ar or nitrogen N₂. Preferably, the entrainment and confinement gases (chimney, reactor windows) are argon Ar. The confinement flow rates are between 0 and 5,000 cm³·min⁻¹, preferably 0 cm³·min⁻¹ for the confinement at the level of the chimney and 3,000 cm³·min⁻¹ for the confinement at the level of the visibility windows. The entrainment gas flow rate is between 50 and 10,000 cm³·min⁻¹, preferably 2,000 cm³·min⁻¹.

Within the reaction chamber, a CO₂ infrared laser beam with a wavelength of 10.6 μm and a power of up to 2,800 W is emitted orthogonally to the mixture of precursors, transported in the form of fine droplets. Ideally, the laser power delivered to the reaction area is between 100 and 900 W and of the order of 670 W for the synthesis of TiO₂ and Cu/TiO₂. A gas absorbing the laser radiation, preferably ethylene C₂H₄, may also be added in a flow rate range from 0 to 5,000 cm³·min⁻¹. In this example, the flow rate of this gas is set at 800 cm³·min⁻¹. In this case, the laser power absorbed by the precursor aerosol reported on Table 1 is 276 W for TiO₂ and 250 W for Cu/TiO₂. The interaction between the laser beam, the precursor aerosol and possibly the ethylene gas allows the growth of nanoparticles collected on the surface of a filter barrier comprising nanopores. It should be noted that the use of ethylene for synthesis is optional and that it is possible to synthesize TiO₂ and Cu/TiO₂ materials without it.

The nano-powders synthesized by said method are then calcined in a tubular furnace via an air reactor to remove the amorphous carbon from the precursors and possibly from the ethylene gas if ethylene is used. The heat treatment applied is, for example, a temperature of 450° C. under air flow at 100 mL·min⁻¹ for a period ranging from 3 to 6 hours—until the amorphous carbon is virtually or completely eliminated, in this case, for example, 6 hours.

The main physico-chemical characteristics of the TiO₂ and Cu/TiO₂ photocatalysts synthesized by laser pyrolysis are shown at Table 2.

TABLE 2

| | Copper content | S_{BET} | % m | % m |
|---|---|---|---|---|
| Material | (% m ICP) | (m² · g⁻¹) | Anatase | Rutile |
| TiO₂ (outside | — | 81 | 72 | 28 |
| the invention) | | | | |
| Cu/TiO₂ | 1.91 | 40 | 53 | 47 |

### Physico-Chemical Characteristics of Photocatalysts Synthesized by Laser Pyrolysis

It should be noted that the copper content in the Cu/TiO₂ material synthesized by laser pyrolysis, determined by inductively coupled plasma spectrometry (ICP), is 1.91% by mass, which is very similar to the percentage introduced into the pyrosol (2.00% by mass). Transmission electron microscopy (TEM) images of TiO₂ and Cu/TiO₂ nanoparticles obtained according to the present method show that the size of the nanoparticles is between 5 and 25 nm for TiO₂ and between 10 and 70 nm for Cu/TiO₂.

Scanning transmission electron microscopy (STEM) and EDX images of Cu/TiO₂ nanoparticles of the invention highlight copper/copper oxide clusters on the TiO₂ surface, with a diameter of between 1 and 3 nm.

### Synthesis by Impregnation on a TiO₂ Support:

The metal (oxide)/TiO₂ photocatalysts of the invention may also be synthesized by impregnation of metal via a metal precursor on a TiO₂ support. This TiO₂ support may be commercial or obtained by laser pyrolysis, such as the TiO₂ described above. An example is given below with copper as the metal.

500 mg of TiO₂ obtained by laser pyrolysis was dissolved in 50 mL of distilled water to which was added 43.3 mg (2.00 wt. %) of a copper precursor, copper acetylacetonate Cu(acac)₂ (purity=97%). The metal precursor, in the case of copper, is not limited to this compound and may be, for example, copper acetate (anhydrous or hydrated) or copper nitrate. Optionally, one or more organic solvents such as ethanol may be added and the precursors dispersed in an ultrasonic bath. Once the precursors have dissolved completely, the mixture is transferred to a 50 mL flat-bottomed flask and heated to 70° C. in a water bath. The stirred mixture is evaporated over a period of 12 hours using a magnetic bar, and the residual powder is dried in an oven at 120° C.

The impregnated powder is then calcined at 450° C. for 6 hours in a reactor in a tubular oven under a flow of air at 100 mL·min⁻¹.

The main physico-chemical characteristics of the TiO₂ support obtained by laser pyrolysis are described and those of the Cu_{IMP}/TiO₂ photocatalyst synthesized by impregnation are presented in the following Table 3.

TABLE 3

| | Copper content | S_{BET} | % m | % m |
|---|---|---|---|---|
| Material | (% m ICP) | (m² · g⁻¹) | Anatase | Rutile |
| TiO₂ (outside | — | 81 | 72 | 28 |
| the invention) | | | | |
| CU_{IMP}/TIO₂ | 2.15 | 37 | 66 | 34 |

Physico-Chemical Characteristics of TiO₂ and Cu_{IMP}/TiO₂ Photocatalysts

It should be noted that the copper content in the Cu/TiO₂ material synthesized by impregnation, determined by inductively coupled plasma spectrometry (ICP), is 2.15% m, which is very similar to the percentage (2.00% m) introduced into the flask for impregnation of the TiO₂ support.

### Example 3: Photocatalysis of Propanoic Acid

The photocatalysts TiO₂ (reference outside the invention) and Cu/TiO₂ (example 2, first part) were introduced into the photocatalytic reactor mentioned above (FIG. 1) at a concentration of 0.5 g·L⁻¹ with 1 vol % propanoic acid in 100 mL of aqueous solution. A flow of argon fixed at 70 mL/min for 6 hours drove the air out of the photocatalytic reactor and replaced it with a neutral argon atmosphere. After complete purging, the argon flow is stopped and the photocatalytic reactor is isolated. The photo-produced gaseous compounds were taken from the headspace of the reactor and sent to the GC/FID and GC/PDHID.

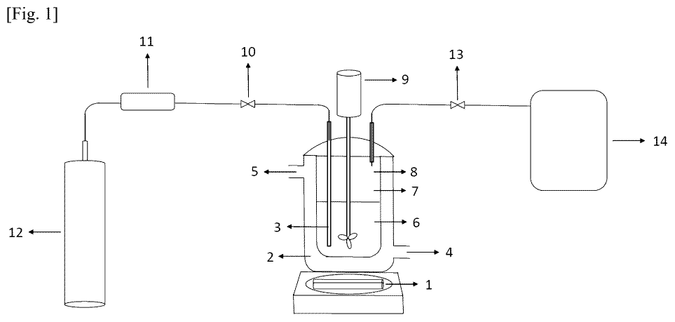

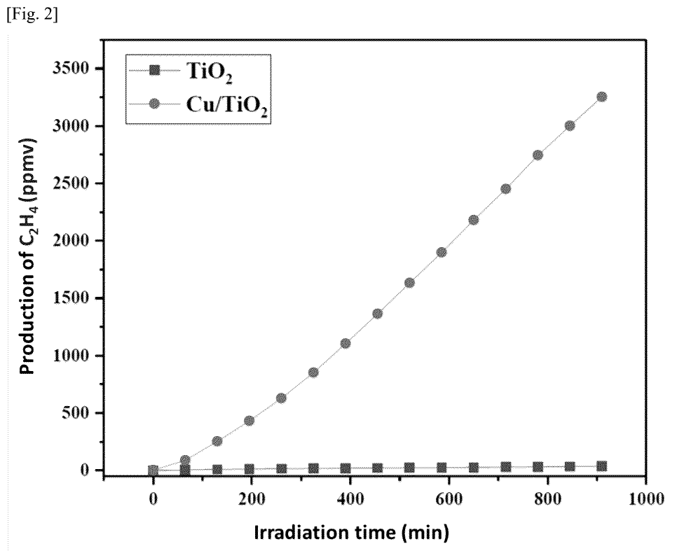

FIG. 2 shows the ethylene produced from propanoic acid (1 vol % in H₂O) for 910 minutes under UVA irradiation centered at 370 nm with TiO₂ and Cu/TiO₂ photocatalysts. The ethylene obtained by photocatalysis with TiO₂ reached a production rate of 2 ppmv/h, and 214 ppmv/h with Cu/TiO₂. This production is linear and the photocatalyst does not lose activity during the 910 minutes of irradiation.

The following Table 4 shows the hourly gas productions obtained for this reaction as well as the selectivity, calculated according to the quotient [compound]/Σ[CₓH_{y}O_{z}] with x and y ≥1 after 910 minutes of irradiation.

TABLE 4

| | | C₂H₄ | | C₂H₆ | | C₄H₁₀ |
|---|---|---|---|---|---|---|
| | | (ppmv/h), | | (ppmv/h), | | (ppmv/h), |
| | | selectivity | | selectivity | | selectivity |
| Material | Summary | (%) | | (%) | | (%) |
| TiO₂ (outside | Laser | 2 | (1%) | 163 | (96%) | 3 (2%) |
| the invention) | pyrolysis | | | | | |
| Cu/TiO₂ | Laser | 214 | (91%) | 18 | (8%) | 2 (1%) |
| | pyrolysis | | | | | |

Hourly production (ppmv/h) and selectivity (%) of photogases produced from 1 vol % propanoic acid under argon by TiO₂ and Cu/TiO₂ synthesized by laser pyrolysis

The ethylene yield, calculated by the ratio [C₂H₄/CO₂] considering that a photodegraded propanoic acid molecule forms an ethane radical and a CO₂ molecule and that an ethane radical may form an ethylene or ethane molecule, is 1.0% for TiO₂ and 85.0% for Cu/TiO₂ after 910 minutes of irradiation.

Similarly, the photocatalysis was performed using impregnated photocatalysts (example 2, second part).

The Cu_{IMP}/TiO₂ impregnated photocatalyst was introduced into the above-mentioned photocatalytic reactor (FIG. 1) at 0.5 g·L⁻¹ with 1 vol % propanoic acid in 100 mL aqueous solution. The synthesis of ethylene by photocatalysis was carried out under the same conditions as described above.

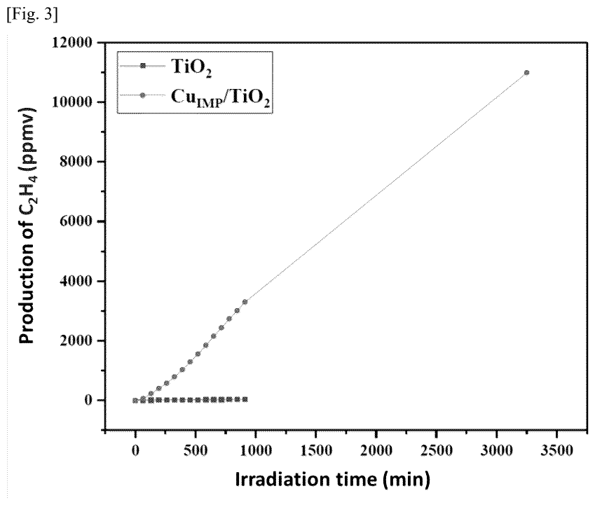

FIG. 3 shows the ethylene produced from propanoic acid (1 vol % in H₂O) under UVA irradiation centered at 370 nm with the photocatalysts TiO₂ (obtained by laser pyrolysis, 910 min irradiation) and Cu_{IMP}/TiO₂ (obtained by impregnation of the above-mentioned TiO₂ support, for 3,250 minutes, i.e. more than 54 hours irradiation). Ethylene synthesized by photocatalysis with TiO₂ achieved a production rate of 2 ppmv/h, and 218 ppmv/h with Cu_{IMP}/TiO₂. This production is linear and the photocatalyst does not lose activity over the 3,250 minutes of irradiation.

The following Table 5 shows the hourly gas productions obtained for this reaction as well as the selectivity, calculated according to the quotient [compound]/Σ[CₓH_{y}O_{z}] with x and y ≥1 after 910 minutes of irradiation.

TABLE 5

| | | C₂H₄ | | C₂H₆ | | C₄H₁₀ |
|---|---|---|---|---|---|---|
| | | (ppmv/h), | | (ppmv/h), | | (ppmv/h), |
| | | selectivity | | selectivity | | selectivity |
| Material | Summary | (%) | | (%) | | (%) |
| TiO₂ (outside | Laser | 2 | (1%) | 163 | (96%) | 3 (2%) |
| the invention) | pyrolysis | | | | | |
| Cu/TiO₂ | Impreg- | 218 | (92%) | 16 | (7%) | 2 (1%) |
| | nation | | | | | |

Hourly Production (ppmv/h) and Selectivity (%) of Photogases Produced From 1 vol % Propanoic Acid Under Argon by TiO₂ Synthesized by Laser Pyrolysis and Cu_{IMP}/TiO₂

The ethylene yield was 1.0% for TiO₂ and 86.5% for Cu_{IMP}/TiO₂ after 910 minutes of irradiation.

### Example 4: Photocatalysis of Ethanol

An ethanol concentration of 1% by mass (i.e. 1.27% by volume) was used. The 2% Cu/TiO₂ catalyst was introduced at 0.5 g_{cat}/L into the resulting 100 mL solution under an inert atmosphere (argon). Still under an inert atmosphere, the UV irradiation (UVA centered at 370 nm) was carried out for around 650 min at room temperature (20-25° C.). The gaseous products were monitored in the same way as for example 3, i.e. with kinetic monitoring by GC-FID and GC-PDHID, and with identification and calibration of the products using standards.

### Results:

After 600 minutes of irradiation, H₂, CO₂, ethylene C₂H₄, acetaldehyde CH₃CHO, methane CH₄ and traces of CO and ethane were obtained.

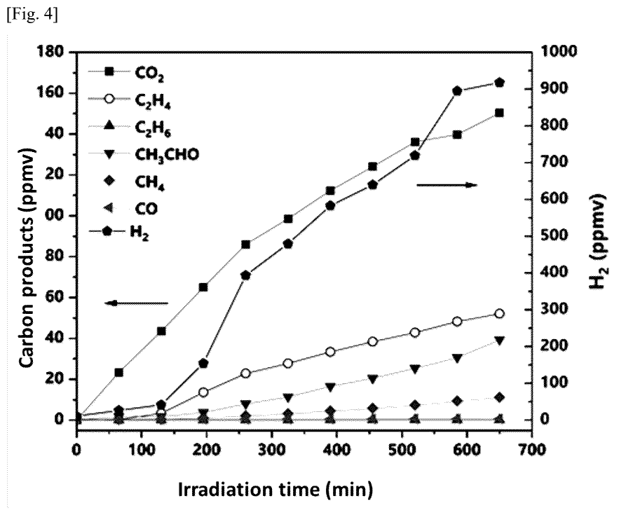

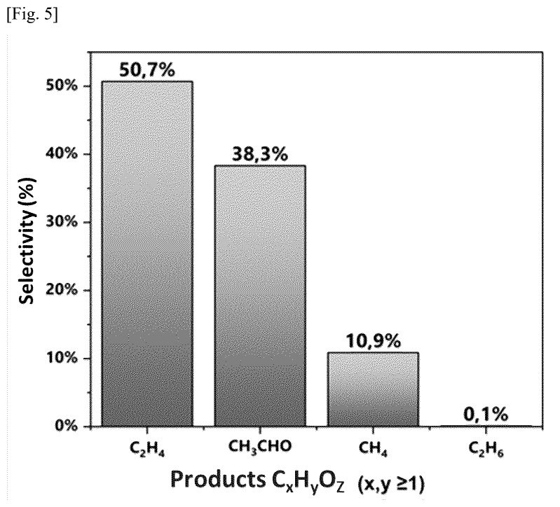

FIG. 4 shows the formation of these species over the irradiation time, and FIG. 5 the selectivity obtained.

The results show an excellent selectivity for ethylene compared with the other CₓH_{y}O_{z} products (x, y≥1). The molar selectivity of ethylene and acetaldehyde is 51% and 38% respectively. Ethylene is therefore the majority CₓH_{y}O_{z} (x, y≥1) product.

Neither acetone nor ethyl acetate was detected.

1. A use of particles consisting of or comprising TiO₂ bearing on at least part of their surface a metal M and/or an oxide of metal M, M being chosen from the group comprising Cu, Zn, Fe, Mo, W and Ni, for obtaining at least one alkene by photocatalysis from at least one carboxylic acid of formula (I) Rₐ—COOH, and/or at least one alcohol of formula (II) R_{b}—OH, wherein Rₐ and R_{b} are independently chosen from linear, branched or cyclic alkyl groups, optionally substituted with at least one group X chosen from arenes, X being in particular a phenyl group.

2. A method for obtaining at least one alkene from at least one carboxylic acid of formula (I) Rₐ—COOH, and/or from at least one alcohol of formula (II) R_{b}—OH, wherein Rₐ and R_{b} are independently chosen from linear, branched or cyclic alkyl groups, optionally substituted with at least one group X chosen from arenes, comprising a step (i) of photocatalysis by UV and/or visible irradiation of at least one carboxylic acid and/or at least one alcohol in the presence of a catalyst consisting of or comprising particles consisting of or comprising TiO₂ bearing on at least part of their surface a metal M and/or an oxide of metal M, M being chosen from the group comprising Cu, Zn, Fe, Mo, W and Ni.

3. The method according to claim 2, wherein:
the largest number-average dimension of the particles consisting of or comprising TiO₂ is between 1 and 100 nm; and/or
the particles consisting of or comprising TiO₂ are spherical, spheroidal, rod-shaped, wire-shaped, tubular and/or platelet-shaped, the particles optionally being organized in chains.

4. The method according to claim 2, wherein the TiO₂ is in the form of anatase, rutile and/or brookite.

5. The method according to claim 2, wherein:
the content of metal M and/or oxide of metal M relative to TiO₂ is between 0.01 and 50% by mass; and/or
the metal M and/or the oxide of metal M are present, at least on the surface of the particles consisting of or comprising TiO₂, in the form of particles whose largest number-average particle size is between 0.1 and 50 nm.

6. The method according to claim 2, for obtaining at least one alkene from at least one carboxylic acid selected from propanoic acid, acetic acid, a phenylpropanoic acid, n-butyric acid, n-valeric acid, or pivalic acid, or from at least one alcohol, selected from ethanol or cyclohexanol, and/or the at least one alkene is ethylene.

7. The method according to claim 2, wherein step (i) is carried out:
under an atmosphere comprising by volume less than 1% oxygen;
in an inert gas atmosphere; and/or
under a continuous flow of inert gas, the flow being between 1 and 500 mL/min; or
in the absence of a continuous flow of inert gas.

8. The method according to claim 2, wherein the at least one carboxylic acid and/or the at least one alcohol is present within a composition additionally comprising a solvent, the solvent being water, the concentration of alcohol or alcohols and/or carboxylic acid or acids in the composition being greater than or equal to 0.0001% by volume, or wherein the at least one carboxylic acid and/or the at least one alcohol is not in the presence of a solvent.

9. The method according to claim 2, wherein the catalyst is present in the composition comprising the at least one carboxylic acid and/or the at least one alcohol and the solvent, or, in the absence of solvent, in the at least one carboxylic acid and/or the at least one alcohol, at a concentration of between 0.01 and 50 g/L.

10. The method according to claim 2, wherein:
step (i) is performed at a temperature comprised from 10 to 200° C.; and/or
the irradiation is a UV-A, UV-B, UV-C and/or visible irradiation.

11. The method according to claim 3, wherein the largest number-average dimension of the particles consisting of or comprising TiO₂ is between 5 and 70 nm.

12. The method according to claim 2, wherein the TiO₂ is in the form of anatase, rutile, or a mixture of anatase and rutile.

13. The method according to claim 12, wherein the TiO₂ is in the form of a mixture of anatase and rutile, and wherein the anatase/rutile ratio is between 0.80 and 2.33.

14. The method according to claim 5, wherein:
the content of metal M and/or oxide of metal M relative to TiO₂ is selected from between 0.1 and 5% by mass, about 2% by mass, or more than 0.01% and less than 2% by mass; and/or
the metal M and/or the oxide of metal M are present, at least on the surface of the particles consisting of or comprising TiO₂, in the form of particles whose largest number-average particle size is between 0.5 and 10 nm or between 1 and 3 nm.

15. The method according to claim 2, wherein for obtaining at least one alkene from at least one carboxylic acid, the at least one carboxylic acid is propanoic acid, acetic acid, a phenylpropanoic acid, n-butyric acid, n-valeric acid, or pivalic acid, or from at least one alcohol, wherein the at least one alcohol is ethanol or cyclohexanol.

16. The method according to claim 7, wherein step (i) is carried out:
under an atmosphere comprising by volume less than 0.1% oxygen;
in a nitrogen, helium and/or argon atmosphere; and/or
under a continuous flow of a nitrogen, helium and/or argon atmosphere, the flow being between 50 and 70 mL/min; or
in the absence of a continuous flow of inert gas.

17. The method according to claim 8, wherein the at least one carboxylic acid and/or the at least one alcohol is present within a composition additionally comprising a solvent in solution, the solvent being water, the concentration of alcohol or alcohols and/or carboxylic acid or acids in the composition being in particular greater than or equal to 0.01% by volume, and/or less than about 1.00% by volume, or wherein the at least one carboxylic acid and/or the at least one alcohol is not in the presence of a solvent.

18. The method according to claim 9, wherein the catalyst is present in the composition comprising the at least one carboxylic acid and/or the at least one alcohol and the solvent, or, in the absence of solvent, in the at least one carboxylic acid and/or the at least one alcohol, at a concentration of about 0.5 g/L.

19. The method according to claim 10, wherein:
step (i) is performed at a temperature selected from a range of about 20 to about 40° C., about 40 to 200° C., about 40 to 150° C., or about 40 to 100° C.; and/or
the irradiation is a UV-A.
